(19) United States
(12) Patent Application Publication
(10) Pub. No.: US 2026/0256750 A1
(43) Pub. Date: Sep. 3, 2026

(54) DIHYDROBERBERINE OR ITS DERIVATIVES FOR REGULATING OR IMPROVING MOODS

(71) Applicant: NANJING NUTRABUILDING BIO-TECH CO., LTD., Nanjing (CN)

(72) Inventors: Ronghua YI, Nanjing (CN); Shawn WELLS, Frisco, TX (US); Kylin LIAO, Nanjing (CN)

(21) Appl. No.: 19/655,680

(22) Filed: Apr. 22, 2026

Related U.S. Application Data
(63) Continuation-in-part of application No. PCT/CN2024/126353, filed on Oct. 22, 2024.

(30) Foreign Application Priority Data
Oct. 25, 2023 (WO) PCT/CN2023/126414

(51) Int. Cl. A61K 31/4375; A61P 25/24; A61P 25/28

(52) U.S. Cl. CPC A61K 31/4375; A61P 25/24; A61P 25/28

(57) ABSTRACT

The present invention provides novel compositions and methods for preventing or alleviating fatigue, enhancing concentration, refreshing and awakening mind, improving energy, regulating or improving moods in a subject. The composition may comprise dihydroberberine (DHB), or a pharmaceutically acceptable salt, polymer, ester, acid, or an analog or derivative thereof.

### CROSS REFERENCE TO RELATED APPLICATION

This application is a continuation-in-part application of International Patent Application No. PCT/CN2024/126353, filed on October 22, 2024, which claims the priority of the International Patent Application No. PCT/CN2023/126414, filed on October 25, 2023, the contents of all of which are incorporated herein by reference in their entirety.

### BACKGROUND

Mood disorders are common, chronic, recurrent mental phenomena that affect millions of individuals’ feelings, motivation, behavior, thoughts, and sense of well-being worldwide (de Zwart, P.L., B.F. Jeronimus, and P. de Jonge, Empirical evidence for definitions of episode, remission, recovery, relapse and recurrence in depression: a systematic review. Epidemiology and Psychiatric Sciences, 2019. 28(5): p. 544-562). The primary mood disorders are major depressive disorder and bipolar disorder. Globally, about 300 million people, i.e., 4.4% of the world’s population, suffer from depression (Global Burden of Disease Study 2015).

Most persons with mood disorders receive some benefits from available treatments. However, full remission of clinical symptoms is rarely achieved owing to complex pathophysiology. Moreover, many classes of antidepressants cause distressing and serious side effects, such as irritability, akathisia, drowsiness, dryness of mouth, headache, nausea, and sexual dysfunction. There is, therefore, an urgent need to develop alternative and effective products with fewer side effects, which reduce or even combat the phenomenon of mood disorders may be appreciated.

Many studies have been shown that the intestinal microbiota influences and modulates the nervous system shape and function, known as the “gut-brain axis”, and the nervous system is responsible for behaviors and moods, like depression (Dalile, B., et al., The role of short-chain fatty acids in microbiota-gut-brain communication. Nature reviews. Gastroenterology & hepatology, 2019. 16(8): p. 461-478). Furthermore, studies have demonstrated the brain‐derived neurotrophic factor (BDNF) level is linked to moods (Zhou, Y., et al., Changes in element accumulation, phenolic metabolism, and antioxidative enzyme activities in the red-skin roots of Panax ginseng. Journal of Ginseng Research, 2017. 41(3): p. 307-315). In this invention, we found that DHB could be considered as a promising and attractive ingredient for preventing or alleviating fatigue, enhancing concentration, refreshing and awakening mind, improving energy, regulating or improving moods by shaping and mediating the function of the “gut-brain axis”.

### SUMMARY

In a first aspect, the present invention provides a method for preventing or alleviating fatigue, enhancing concentration, refreshing and awakening mind, improving energy, regulating or improving moods in a subject, comprising administrating to the subject a composition comprising: dihydroberberine (DHB), or a pharmaceutically acceptable salt, polymer, ester, acid, or an analog or derivative thereof.

In some embodiments, the moods comprise anxiety, stress, or depression, obsessive-compulsive disorder, and bipolar disorder. Mood disorders are common, chronic, recurrent mental phenomena that affect millions of individuals’ feelings, motivation, behavior, thoughts, and sense of well-being worldwide.

In some embodiments, the preventing or alleviating fatigue, enhancing concentration, refreshing and awakening mind, improving energy, regulating or improving moods are achieved by improving the brain function, shaping and mediating the function of “gut-brain axis”, upregulating the biosynthesis of L-dopa, mitigating the depressive-like behaviors, elevating brain-derived neurotrophic factor (BDNF) level.

In some embodiments, dihydroberberine (DHB), or a pharmaceutically acceptable salt, polymer, ester, acid, or an analog or derivative thereof is administered in an effective amount.

In some embodiments, a proportion of the dihydroberberine (DHB) is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the dihydroberberine (DHB), or a pharmaceutically acceptable salt, polymer, ester, acid, or an analog or derivative thereof is administrated at a daily dose of 10-2000 mg. In some embodiments, the dihydroberberine (DHB), or a pharmaceutically acceptable salt, polymer, ester, acid, or an analog or derivative thereof is administrated at a daily dose of 10-1500 mg, 20-1000 mg, 10-800 mg, 20-600 mg, 30-300 mg, 40-200 mg, 40-100 mg. In some embodiments, the daily dose is administered in divided doses or a single dose. In some embodiments, the administration is at least once a day or more times a day. In some embodiments, the administration is at least 7 days and above in one period. In some embodiments, the administration lasts for a period of time. In some embodiments, the administration may last for one week, two weeks, three weeks, four weeks, two months, three months, four months or longer.

In some embodiments, the dihydroberberine (DHB), or a pharmaceutically acceptable salt, polymer, ester, acid, or an analog or derivative thereof is administrated in an amount of 0.1 μM to 1 M or 0.01 to 50.0wt% (w/w). In some embodiments, the dihydroberberine (DHB), or a pharmaceutically acceptable salt, polymer, ester, acid, or an analog or derivative thereof may be administrated in an amount of 0.1 μM to 500 μM, 1 μM to 500 μM, 1 μM to 5 mM, 1 μM to 500 mM, 5 μM to 500 μM, 5 μM to 5 mM, 5 μM to 100 mM, 5 μM to 500 mM, 50 μM to 500 μM, 50 μM to 5 mM. In some embodiments, the dihydroberberine (DHB), or a pharmaceutically acceptable salt, polymer, ester, acid, or an analog or derivative thereof may be administrated in an amount of 0.05% to 45%, 0.05% to 40%, 0.05% to 30%, 0.05% to 25%, 0.1% to 40%, 0.1% to 30%, 0.1% to 25%, 0.1% to 15%, 0.5% to 30%, 0.5% to 20%, 0.5% to 10%, 1% to 35%, 1% to 25%, 1% to 10%, 2% to 25%, 2% to 10%, 5% to 9%, 1% to 7%, 2% to 7%, 0.05% to 7%, 0.05% to 9wt% (w/w).

In some embodiments, the subject is human or cattle or pet. In some embodiments, the subject is human.

In some embodiments, the composition is prepared as a food, a drink, a supplement, a biochemical composition, or a nutraceutical composition.

In some embodiments, the administration is through various routes selected from oral, intravenous injection, intramuscular injection, intraperitoneal injection, external use, or sublingual application.

In some embodiments, the composition is formulated in solutions, aqueous suspensions, liquid suspensions, parenteral solutions, injections, microemulsion, (micro)capsules, drops, granules, liquids, powders, aerosols, tonics, syrups, tablets, pills, film, functionalized foods, beverages, toothpaste, nourishments, snacks, gums, bars, sugars, and sublingual articles.

In some embodiments, compared with non-supplement group, L-dopa level in group supplemented with the dihydroberberine (DHB), or a pharmaceutically acceptable salt, polymer, ester, acid, or an analog or derivative thereof improves. In some embodiments, compared with non-supplement group, L-dopa level in group supplemented with the dihydroberberine (DHB), or a pharmaceutically acceptable salt, polymer, ester, acid, or an analog or derivative thereof is about 110%, 120%, 130%, 140%, 150%, 180%, 210%, 240%, 270%, 300%, 340%.

In some embodiments, compared with non-supplement group, dopamine level in group supplemented with the dihydroberberine (DHB), or a pharmaceutically acceptable salt, polymer, ester, acid, or an analog or derivative thereof improves. In some embodiments, compared with non-supplement group, dopamine level in group supplemented with the dihydroberberine (DHB), or a pharmaceutically acceptable salt, polymer, ester, acid, or an analog or derivative thereof is about 110%, 120%, 130%, 140%, 150%, 175%, 190%, 210%, 235%.

In some embodiments, BDNF level in PFC improves in group supplemented with the dihydroberberine (DHB), or a pharmaceutically acceptable salt, polymer, ester, acid, or an analog or derivative thereof, relative to non-supplement group. In some embodiments, BDNF level in PFC in group supplemented with the dihydroberberine (DHB), or a pharmaceutically acceptable salt, polymer, ester, acid, or an analog or derivative thereof is about 103%, 105%, 108%, 110%, 115%, 120%, 123%, relative to non-supplement group.

In some embodiments, BDNF level in HPC improves in group supplemented with the dihydroberberine (DHB), or a pharmaceutically acceptable salt, polymer, ester, acid, or an analog or derivative thereof, relative to non-supplement group. In some embodiments, BDNF level in HPC in group supplemented with the dihydroberberine (DHB), or a pharmaceutically acceptable salt, polymer, ester, acid, or an analog or derivative thereof is about 105%, 108%, 110%, 120%, 130%, 140%, 150%, 155%, relative to non-supplement group.

In a second aspect, the present invention provides a composition comprising an effective amount of dihydroberberine (DHB), or a pharmaceutically acceptable salt, polymer, ester, acid, or an analog or derivative thereof, for preventing or alleviating fatigue, enhancing concentration, refreshing and awakening mind, improving energy, regulating or improving moods in a subject.

In some embodiments, the preventing or alleviating fatigue, enhancing concentration, refreshing and awakening mind, improving energy, regulating or improving moods are achieved by improving the brain function, shaping and mediating the function of “gut-brain axis”, upregulating the biosynthesis of L-dopa, mitigating the depressive-like behaviors, elevating brain-derived neurotrophic factor (BDNF) level.

In some embodiments, a proportion of the dihydroberberine (DHB) is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the dihydroberberine (DHB), or a pharmaceutically acceptable salt, polymer, ester, acid, or an analog or derivative thereof is administrated at a daily dose of 10-2000 mg. In some embodiments, the dihydroberberine (DHB), or a pharmaceutically acceptable salt, polymer, ester, acid, or an analog or derivative thereof is administrated at a daily dose of 10-1500 mg, 20-1000 mg, 10-800 mg, 20-600 mg, 30-300 mg, 40-200 mg, 40-100 mg. In some embodiments, the daily dose is administered in divided doses or a single dose. In some embodiments, the administration is at least once a day or more times a day. In some embodiments, the administration is at least 7 days and above in one period.

In some embodiments, the dihydroberberine (DHB), or a pharmaceutically acceptable salt, polymer, ester, acid, or an analog or derivative thereof is administrated in an amount of 0.1 μM to 1 M or 0.01 to 50.0wt% (w/w). In some embodiments, the dihydroberberine (DHB), or a pharmaceutically acceptable salt, polymer, ester, acid, or an analog or derivative thereof may be administrated in an amount of 0.1 μM to 500 μM, 1 μM to 500 μM, 1 μM to 5 mM, 1 μM to 500 mM, 5 μM to 500 μM, 5 μM to 5 mM, 5 μM to 100 mM, 5 μM to 500 mM, 50 μM to 500 μM, 50 μM to 5 mM. In some embodiments, the dihydroberberine (DHB), or a pharmaceutically acceptable salt, polymer, ester, acid, or an analog or derivative thereof may be administrated in an amount of 0.05% to 45%, 0.05% to 40%, 0.05% to 30%, 0.05% to 25%, 0.1% to 40%, 0.1% to 30%, 0.1% to 25%, 0.1% to 15%, 0.5% to 30%, 0.5% to 20%, 0.5% to 10%, 1% to 35%, 1% to 25%, 1% to 10%, 2% to 25%, 2% to 10%, 5% to 9%, 1% to 7%, 2% to 7%, 0.05% to 7%, 0.05% to 9wt% (w/w).

In some embodiments, the subject is human or cattle or pet. In some embodiments, the subject is human.

In some embodiments, the composition is prepared as a food, a drink, a supplement, a biochemical composition, or a nutraceutical composition.

In some embodiments, the composition is formulated in solutions, aqueous suspensions, liquid suspensions, parenteral solutions, injections, microemulsion, (micro)capsules, drops, granules, liquids, powders, aerosols, tonics, syrups, tablets, pills, film, functionalized foods, beverages, toothpaste, nourishments, snacks, gums, bars, sugars, and sublingual articles.

In a third aspect, the present invention provides use of a composition in a preparation of food, drink, supplement, biochemical composition, or nourishment for preventing or alleviating fatigue, enhancing concentration, refreshing and awakening mind, improving energy, regulating or improving moods in a subject, wherein the composition comprises dihydroberberine (DHB), or a pharmaceutically acceptable salt, polymer, ester, acid, or an analog or derivative thereof.

In some embodiments, the moods comprise anxiety, stress, or depression, obsessive-compulsive disorder, and bipolar disorder.

In some embodiments, the preventing or alleviating fatigue, enhancing concentration, refreshing and awakening mind, improving energy, regulating or improving moods are achieved by improving the brain function, shaping and mediating the function of “gut-brain axis”, upregulating the biosynthesis of L-dopa, mitigating the depressive-like behaviors, elevating brain-derived neurotrophic factor (BDNF) level.

In some embodiments, a proportion of the dihydroberberine (DHB) is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the dihydroberberine (DHB), or a pharmaceutically acceptable salt, polymer, ester, acid, or an analog or derivative thereof is administrated at a daily dose of 10-2000 mg. In some embodiments, the dihydroberberine (DHB), or a pharmaceutically acceptable salt, polymer, ester, acid, or an analog or derivative thereof is administrated at a daily dose of 10-1500 mg, 20-1000 mg, 10-800 mg, 20-600 mg, 30-300 mg, 40-200 mg, 40-100 mg. In some embodiments, the daily dose is administered in divided doses or a single dose. In some embodiments, the administration is at least once a day or more times a day. In some embodiments, the administration is at least 7 days and above in one period.

In some embodiments, the dihydroberberine (DHB), or a pharmaceutically acceptable salt, polymer, ester, acid, or an analog or derivative thereof is administrated in an amount of 0.1 μM to 1 M or 0.01 to 50.0wt% (w/w). In some embodiments, the dihydroberberine (DHB), or a pharmaceutically acceptable salt, polymer, ester, acid, or an analog or derivative thereof may be administrated in an amount of 0.1 μM to 500 μM, 1 μM to 500 μM, 1 μM to 5 mM, 1 μM to 500 mM, 5 μM to 500 μM, 5 μM to 5 mM, 5 μM to 100 mM, 5 μM to 500 mM, 50 μM to 500 μM, 50 μM to 5 mM. In some embodiments, the dihydroberberine (DHB), or a pharmaceutically acceptable salt, polymer, ester, acid, or an analog or derivative thereof may be administrated in an amount of 0.05% to 45%, 0.05% to 40%, 0.05% to 30%, 0.05% to 25%, 0.1% to 40%, 0.1% to 30%, 0.1% to 25%, 0.1% to 15%, 0.5% to 30%, 0.5% to 20%, 0.5% to 10%, 1% to 35%, 1% to 25%, 1% to 10%, 2% to 25%, 2% to 10%, 5% to 9%, 1% to 7%, 2% to 7%, 0.05% to 7%, 0.05% to 9wt% (w/w).

In some embodiments, the subject is human or cattle or pet. In some embodiments, the subject is human.

In some embodiments, the administration is through various routes selected from oral, intravenous injection, intramuscular injection, intraperitoneal injection, external use, or sublingual application.

In some embodiments, the composition is formulated in solutions, aqueous suspensions, liquid suspensions, parenteral solutions, injections, microemulsion, (micro)capsules, drops, granules, liquids, powders, aerosols, tonics, syrups, tablets, pills, film, functionalized foods, beverages, toothpaste, nourishments, snacks, gums, bars, sugars, and sublingual articles.

These and other features, aspects, and advantages of the present invention will become better understood with reference to the following description and appended claims.

### BRIEF DESCRIPTION OF THE DRAWINGS

FIG. 1 is a graph of L-dopa levels after DHB or normal saline administration (Groups 1-3).

FIG. 2 is a graph of dopamine levels after DHB or normal saline administration (Groups 1-3).

FIG. 3 shows the results of forced swim test conducted after DHB or normal saline administration (Groups 1-3).

FIG. 4 shows the results of tail suspension test conducted after DHB or normal saline administration (Groups 1-3).

FIG. 5 shows the levels of BDNF in the prefrontal cortex (PFC) and hippocampus (HPC) after DHB or normal saline administration (Groups 1-3).

### DETAILED DESCRIPTION

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” and their conjugations, refer to a situation wherein said terms are used in their non-limiting sense to mean that items following the word are included, but items not specifically mentioned are not excluded. It also encompasses the more limiting verb ‘to consist essentially of’ and ‘to consist of’.

As used herein, the term "effective amount" refers to the amount required to achieve the effect as taught herein. The specific effective dose level for any particular subject will depend upon a variety of factors including the conditions being treated and the severity of the conditions; the specific composition employed; the age, body weight, general health, sex and diet of the subject; the time of administration, route of administration, and rate of excretion of dihydroberberine (DHB), or a pharmaceutically acceptable salt, polymer, ester, acid, or an analog or derivative thereof employed; the duration of the treatment; and like factors well known in the medical arts. For example, it is well known within the skill of the art to start doses of the compound at levels lower than those required to achieve the desired effect and to gradually increase the dosage until the desired effect is achieved.

One of skill in the art recognizes that an amount may be considered “effective” even if the condition is not totally eradicated or prevented, but it or its symptoms and/or effects are improved or alleviated partially in the subject.

As used herein, the term “pharmaceutically acceptable” means pharmaceutically, physiologically, alimentarily, and/or nutritionally acceptable, and refers to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio.

As used herein, the term “mammal” or “subject” may be used interchangeably to refer to any animal to which the presently disclosed methods and compositions may be applied or administered. The animal may have an illness or other disease, but the animal does not need to be sick to benefit from the presently disclosed methods and compositions. As such any animal may apply the disclosed combinations, compositions or kits, or be a recipient of the disclosed methods. “Mammal” includes, without limitation, mice, rats, rabbits, guinea pigs, dogs, cats, sheep, goats, cows, horses, primates, such as monkeys, chimpanzees, and apes, and, in particular, humans. Although the animal subject is preferably a human, the methods and compositions of the invention have application in veterinary medicine.

The dosage of dihydroberberine (DHB), or a pharmaceutically acceptable salt, polymer, ester, acid, or an analog or derivative thereof and/or composition comprising the same may range broadly, depending upon the desired effects and the indication. The dosage may be a single one or a series of two or more given in the course of one or more days, as is needed by the subject. In some embodiments, the compounds are administered for a period of time, for example for a week or more, or for months or years.

As used herein, the term "administration" refers to the process of delivering a disclosed composition or active ingredient to a subject. The compositions of the invention can be administered in a variety of ways, including orally, intragastrically, and parenterally (e.g., intravenous and intraarterial as well as other suitable parenteral routes), in external use, and the like.

As used herein, a “parenteral solution” refers to a solution that can be administered elsewhere in the body than the mouth and alimentary canal. It is not delivered via the intestinal tract. For example, parenteral solution can be delivered intravenously.

As used herein, a “tonic” refers to a medicinal substance taken to give a feeling of vigor or well-being.

As used herein, a “syrup” refers to a thick sticky liquid derived from a sugar-rich plant, for example, sugar cane, corn, and maple.

Multiple techniques of administering a composition exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections.

“Intraperitoneal” as used here means within or administered through the peritoneum. The peritoneum is a thin, transparent membrane that lines the walls of the abdominal (peritoneal) cavity and contains/encloses the abdominal organs such as the stomach and intestines.

As used herein, “sublingual” refers to situated or applied under the tongue.

A “functionalized food composition” includes a food composition that has a potentially positive effect on health beyond basic nutrition.

Any titles or subheadings used herein are for organization purposes and should not be used to limit the scope of embodiments disclosed herein.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

All publications and patent applications cited in this specification are herein incorporated by reference as if each individual publication or patent application were specifically and individually indicated to be incorporated by reference.

### Examples

24 of adult male C57BL/6J mice aged about 11 weeks were housed in groups of five per cage in a 22 ± 2 °C and 45-65% relative humidity environment under a normal light cycle room (12-h light/12-h dark; 8:00 a.m. light on ~8:00 p.m. light off). All mice had free access to food and water. Mice were allowed to adapt to their new housing conditions for 1 week before the onset of the experiments.

### Depressive-like behaviors induction:

Lipopolysaccharide (LPS, Escherichia coli, serotype 055: B5) was used to induce depression-like state in 12-week-old mice. LPS was injected intraperitoneally (i.p.) at a dose of 0.5 mg/kg dissolved in the sterile, endotoxin-free 0.9% saline vehicle. Mice (n=24) were subjected to behavioral tests 24 h after LPS injection. These tests were conducted on consecutive two days in orders as forced swim test (FST) and tail suspension test (TST).

Behavioral tests are as follows:

Forced swim test (FST): Mice were placed in a vertical transparent cylinder (30 cm in height and 12 cm in diameter) containing tap water at 23 ± 1 °C and 20 cm in depth. After 1 min habituation, the immobility time of mice was recorded for the further 5 min. The immobility time was recorded and analyzed by the EthoVision XT video tracking software.

Tail suspension test (TST): Mice were suspended upside down by tails 40 cm above the floor by adhesive tape placed 1 cm form the tail tip. During a 6-min test period, the immobility time was scored for the last 5 min following 1-min habituation by the EthoVision XT video tracking software.

If all mice had manifest behavioral alterations, like the prolonged immobility time in FST and TST, then they were supplemented DHB or saline solution next.

### DHB supplementation:

After successful induction, all mice were randomly divided into 4 groups of 6 animals: (1) non-supplement group (Con, Group 1); mice were gavage (i.g.) administrated with normal saline solution daily for 2 days between 9:30 and 11:30 am and then feed the food. (2) DHB supplement group (DHB, 100 mg/kg, Group 2); mice were gavage (i.g.) administrated with DHB in normal saline solution daily for 2 days between 9:30 and 11:30 am and then feed the food. (3) DHB supplement group (DHB, 200 mg/kg, Group 3); mice were gavage (i.g.) administrated with DHB in normal saline solution daily for 2 days between 9:30 and 11:30 am and then feed the food. The total experimental time is about 2 weeks, including two periods: a one-week adaptation period, a two-day induction period, and a subsequent two-day supplementation period. At the end of the study, mice were anesthetized with isoflurane and perfused with 4% paraformaldehyde solution. The brains were removed and post-fixed overnight, then cryoprotected in 20% sucrose solution, embedded in optimal cutting temperature (OCT) compound, and serial 18 μm sagittal sections were made with a cryostat for the immunohistochemical (IHC) and in situ hybridization (ISH) staining.

### DHB supplementation could improve brain function by upregulating the biosynthesis of L-dopa

Dopamine is a predominant neurotransmitter, and its level in the brain is closely related to brain function. Dopamine is primarily synthesized in brain neuron cells. The major synthetic route for dopamine in humans is the “L-phenylalanine (Phe) →L-tyrosine (Tyr) →(s)-3,4-dihydroxyphenylalanine (levodopa, L-dopa) →dopamine (Phe–Tyr–dopa–dopamine)” metabolic pathway. Therefore, L-dopa and dopamine are essential for brain function.

Some of the bacteria could be viewed as a biotech factory to produce L-dopa/dopamine. Interestingly, bacteria in the human intestine, such as Enterococcus, are producers of dopa/dopamine, providing a possible chemical link between gut and brain. Hence, the gut microbiota is an increasingly recognized factor that influences brain function.

Quantitative analysis of L-dopa and dopamine in brain of mice supplemented with or without DHB by LC-MS/MS. The samples of brains were obtained from each group at 0 and 12 h after DHB or saline administration. The IS solution (benzylamine, 0.1 µg/mL, 10 µL) was added to the brain homogenate (100 μL). After precipitating protein with acetonitrile (300 µL), samples were mixed for 30 s and centrifuged at 21.1 × 10³ × g for 10 min. An aliquot (5 µL) was injected into the LC-MS/MS for analysis.

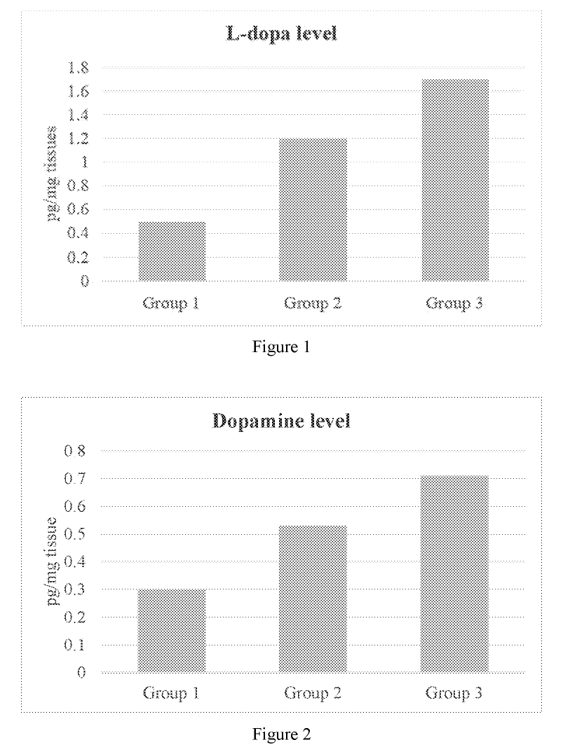

FIG. 1 is a graph of L-dopa levels after DHB or normal saline administration (Groups 1-3). FIG. 2 is a graph of dopamine levels after DHB or normal saline administration (Groups 1-3).

Mice in groups receiving DHB supplementation (Groups 2 and 3) could enhance the biosynthesis of L-dopa in the gut microbiota, relative to mice in group not receiving DHB supplementation (Group 1), indicating that supplementing DHB can improve brain L-dopa/dopamine levels by regulating gut microbiota. Notably, compared with non-supplement group, the higher dose of DHB (Group 3) was about 210% and 230% on the imaging intensity of brain L-dopa and dopamine, respectively. The brain L-dopa and dopamine levels in Group 2 were both not less than 1.5 times that of non-supplement group, and the brain L-dopa and dopamine levels in Group 3 were both more than twice that of non-supplement group. Additionally, the higher dose of DHB (Group 3) can have greater effects on the imaging intensity of brain L-dopa/dopamine than the lower dose of DHB (Group 2). This effect persists for as long as the test protocol is continued.

### DHB supplementation could improve moods and mitigate depressive-like behaviors in mice

After DHB or normal saline administration (Groups 1-3, n=18), the behavioral tests were conducted on consecutive two days in orders as Forced Swimming Test (FST) and Tail suspension test (TST).

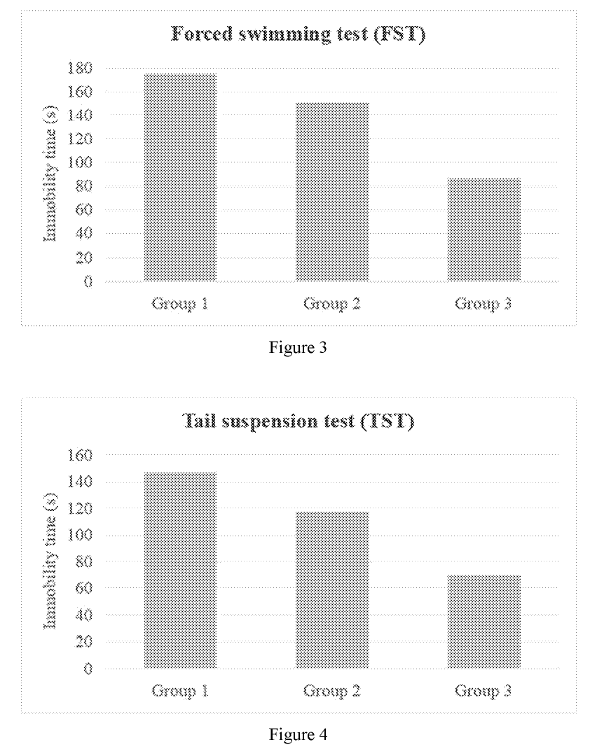

FIG. 3 shows the results of forced swim test conducted after DHB or normal saline administration (Groups 1-3). FIG. 4 shows the results of tail suspension test conducted after DHB or normal saline administration (Groups 1-3).

Over the course of the study, mice in groups receiving DHB supplementation (Groups 2 and 3) could shorten the immobility time, relative to mice in group not receiving DHB supplementation (Group 1), indicating that supplementing DHB can achieve the beneficial effects of moods and depressive-like behaviors. Especially, the higher dose of DHB administered in Group 3 was 50% greater than that of Group 1. Additionally, the higher dose of DHB can have greater effects on moods and behaviors than the lower dose of DHB. This effect persists for as long as the test protocol is continued.

### DHB supplementation could elevate the BDNF level

BDNF level of the cerebrospinal fluid was measured by ELISA kit according to the manufacturer’s protocol.

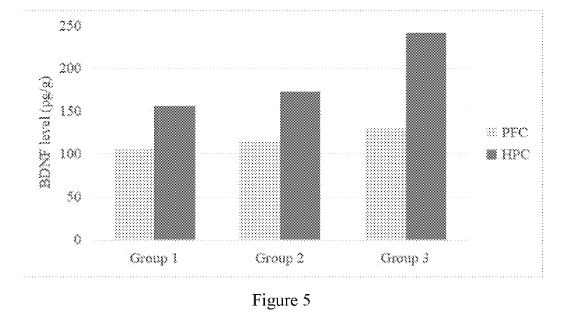

FIG. 5 shows the levels of BDNF in the prefrontal cortex (PFC) and hippocampus (HPC) after DHB or normal saline administration (Groups 1-3). It is observed that mice in groups receiving DHB supplementation (Groups 2 and 3) could restore the decreased levels of BDNF in the PFC and HPC in mice, relative to mice in the group not receiving DHB supplementation (Group 1), indicating that supplementing DHB can achieve beneficial effects on moods and behaviors. It is worth noting that the higher dose of DHB (Group 3) demonstrated BDNF levels of approximately 124% and 155% over Group 1 in the PFC and HPC, respectively. Additionally, the higher dose of DHB can elevate the BDNF level in the PFC and HPC more significantly than the lower dose of DHB. This effect persists for as long as the test protocol is continued.

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

What is claimed is:

1. A method for preventing or alleviating fatigue, enhancing concentration, refreshing and awakening mind, improving energy, regulating or improving moods in a subject, comprising administrating to the subject a composition comprising: dihydroberberine (DHB), or a pharmaceutically acceptable salt, polymer, ester, acid, or an analog or derivative thereof.

2. The method of claim 1, wherein the moods comprise anxiety, stress, or depression, obsessive-compulsive disorder, and bipolar disorder.

3. The method of claim 1, wherein the preventing or alleviating fatigue, enhancing concentration, refreshing and awakening mind, improving energy, regulating or improving moods are achieved by improving the brain function, shaping and mediating the function of “gut-brain axis”, upregulating the biosynthesis of L-dopa, mitigating the depressive-like behaviors, elevating brain-derived neurotrophic factor (BDNF) level.

4. The method of claim 1, wherein a proportion of the dihydroberberine (DHB) is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

5. The method of claim 1, wherein the dihydroberberine, or a pharmaceutically acceptable salt, polymer, ester, acid, or an analog or derivative is administrated at a daily dose of 10-2000 mg.

6. The method of claim 1, wherein the Dihydroberberine (DHB), or a pharmaceutically acceptable salt, polymer, ester, acid, or an analog or derivative thereof is administrated in an amount of 0.1 μM to 1 M or 0.01 to 50.0wt% (w/w).

7. The method of claim 1, wherein the subject is human or cattle or pet.

8. The method of claim 1, wherein the composition is prepared as a food, a drink, a supplement, a biochemical composition, or a nutraceutical composition.

9. The method of claim 1, wherein the administration is through various routes selected from oral, intravenous injection, intramuscular injection, intraperitoneal injection, external use, or sublingual application.

10. The method of claim 1, wherein the composition is formulated in solutions, aqueous suspensions, liquid suspensions, parenteral solutions, injections, microemulsion, (micro)capsules, drops, granules, liquids, powders, aerosols, tonics, syrups, tablets, pills, film, functionalized foods, beverages, toothpaste, nourishments, snacks, gums, bars, sugars, and sublingual articles.

11. The method of claim 1, wherein the dihydroberberine (DHB), or a pharmaceutically acceptable salt, polymer, ester, acid, or an analog or derivative thereof is administered in an effective amount.

12. The method of claim 5, wherein the daily dose is administered in divided doses or a single dose.

13. The method of claim 1, wherein the administration is at least once a day or more times a day.

14. The method of claim 1, wherein the administration is at least 7 days and above in one period.

15. The method of claim 1, wherein the administration lasts for a period of time.

16. The method of claim 1, wherein the subject is human.

17. The method of claim 1, wherein compared with non-supplement group, L-dopa level in group supplemented with the dihydroberberine (DHB), or a pharmaceutically acceptable salt, polymer, ester, acid, or an analog or derivative thereof improves.

18. The method of claim 1, wherein compared with non-supplement group, dopamine level in group supplemented with the dihydroberberine (DHB), or a pharmaceutically acceptable salt, polymer, ester, acid, or an analog or derivative thereof improves.

19. The method of claim 1, wherein BDNF level in PFC improves in group supplemented with the dihydroberberine (DHB), or a pharmaceutically acceptable salt, polymer, ester, acid, or an analog or derivative thereof, relative to non-supplement group.

20. The method of claim 1, wherein BDNF level in HPC improves in group supplemented with the dihydroberberine (DHB), or a pharmaceutically acceptable salt, polymer, ester, acid, or an analog or derivative thereof, relative to non-supplement group.
